(19) United States
(12) Patent Application Publication
(10) Pub. No.: US 2007/0048391 A1
(43) Pub. Date: Mar. 1, 2007

(54) Composition for reduction of scar formation on wound scar

(75) Inventors: Ki Chang Keum, Seoul (KR); Naechoon Yoo, Seoul (KR); Won Min Yoo, Seoul (KR)

Correspondence Address: KING & SPALDING LLP, 1180 PEACHTREE STREET, ATLANTA, GA, 30309-3521, US

(73) Assignee: CambridgeMED, Inc., Cambridge, MA (US)

(21) Appl. No.: 11/210,619

(22) Filed: Aug. 23, 2005

(51) Int. Cl. A61K 31/728; A61K 33/00

(52) U.S. Cl. 424/722; 514/54

(57) ABSTRACT

The present invention relates to a pharmaceutical composition for inhibiting scar formation on wound region which consists of hyaluronic acid and alkalization agent such as sodium bicarbonate, NaOH, and KOH.

### TECHNICAL FIELD

The present invention relates to a composition for reducing scar formation on wound region, which consists of alkalinizing agent and hyaluronic acid and has an, alkaline pH.

### RELATED PRIOR ART

We have showed in. Korea patent No. 0415332 that alkalinization of wound region may induce inactiviation of TGF-β, thereby inhibiting the scar formation. Meanwhile, U.S. Pat. No. 5,731,298 discloses that cross-linked hyaluronic acid may be used for treatment of scars

### DETAILED DESCRIPTION OF THE INVENTION

We have made extensive and intensive researches to develop a medicine with an improved activity in inhibiting scar formation on wound region, and have found that hyaluronic acid compositions having alkaline pHs show the highly greater inhibition of scar formation than each of hyaluronic acid composition having pHs less than 7 or alkalinizing agent individually, that is a synergistic effect in inhibition of scar formation

The present invention relates to a pharmaceutical composition for inhibition of scar formation on wound region which consists essentially of alkalinizing agent and hyaluronic acid and has an alkaline pH. Representative examples of the alkalinizing agent include, but are not limited to, sodium bicarbonate, NaOH and KOH. The pH of the composition is preferably from 8.0 to 9.0, most preferably 8.5.

A composition of the present invention may be formulated into various forms such as injection, ointment, gel, cream, liquid and suspension.

### BRIEF DESCRIPTION OF DRAWINGS

FIG. 1 shows the effect of pH 8.5 sodium bicarbonate solution on inhibiting the in vitro cell growth (None=DPBS solution; Sod-bicar=8.4% sodium bicarbonate injection solution; pH 8.5 Sod-bicar=pH 8.5 sodium bicarbonate injection solution).

FIG. 2 shows the wounded sites on dorsal region of the test rats.

FIG. 3 is a MT staining photograph of the tissue treated in vivo with a sodium bicarbonate solution having pH 7.0.

FIG. 4 is a MT staining photograph of tissues treated in vivo with a sodium bicarbonate solution having pH 8.5.

FIG. 5 shows the effect of hyaluronic acid solution on inhibiting the in vitro cell growth.

FIG. 6 is a MT staining photograph of the tissue treated in vivo with DPBS.

FIG. 7 is a MT staining photograph of the tissue treated in vivo with hyaluronic acid solution.

FIG. 8 shows the effect of a pH 8.5 hyaluronic acid solution on inhibiting the in vitro cell growth.

FIG. 9 is a MT staining photograph of tissues treated in vivo with hyaluronic acid solution.

FIG. 10 is a MT staining photograph of the tissue treated in vivo with pH 8.5 hyaluronic acid solution.

FIG. 11 shows the effect of pH 8.5 sodium bicarbonate solution on inhibiting the in vitro cell growth.

FIG. 12 is a MT staining photograph of the tissue treated in vivo with pH 8.5 sodium bicarbonate solution.

FIG. 13 is a MT staining photograph of the tissue treated in vivo with pH 8.5 hyaluronic acid solution.

### EXAMPLES

The present invention is described more specifically by the following Examples. Examples herein are meant only to illustrate the present invention, but in no way to limit the claimed invention.

### Comparative Example 1

### Alkaline Solution (pH 8.5)

Preparation

Solution A was 8.4% sodium bicarbonate injection solution purchased from Jeil Pharm. Co., Ltd., Seoul, Korea and adjusted to have pH 7.0 by adding 1N NaOH solution and 1N HCl solution. Solution B was sodium bicarbonate solution having pH 8.5 which was prepared by adjusting the pH of the solution A as 8.5 with the addition of 1N NaOH solution and 1N HCl solution.

Experimentation

In Vitro

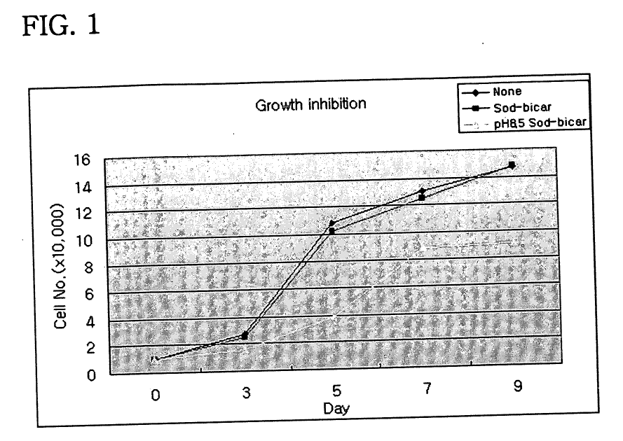

WS1 cells that showed more than 95% viability in trypan blue test were selected, thawed from liquid nitrogen, and then grown in 75 cc flask (T75) to form 80% monolayer (about 6×10⁶/T75) on MEM medium (10% FBS). The detection was performed on 24-well plate at the concentration of 10,000 and was repeated three times on each sample. Each well (10⁴ cells/well) in a 24-well plate containing 3.6 ml of cell suspension was inoculated with solution A or solution B and cultured at 37° C. for 24 hours. The number of cells was counted using hemocytometer 3, 5, 7, and 9 days after the inoculation. The results are shown in FIG. 1.

It can be seen from FIG. 1 that the effect of the solution H on inhibiting cell growth is greater than that of the solution A.

In Vivo

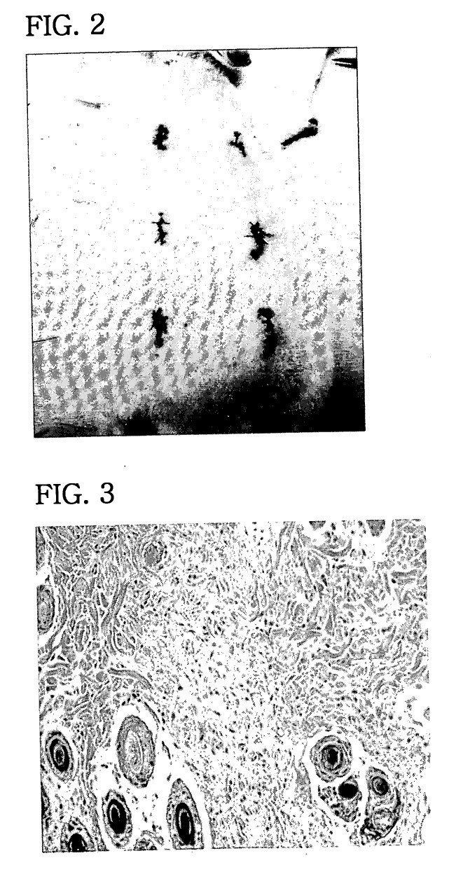

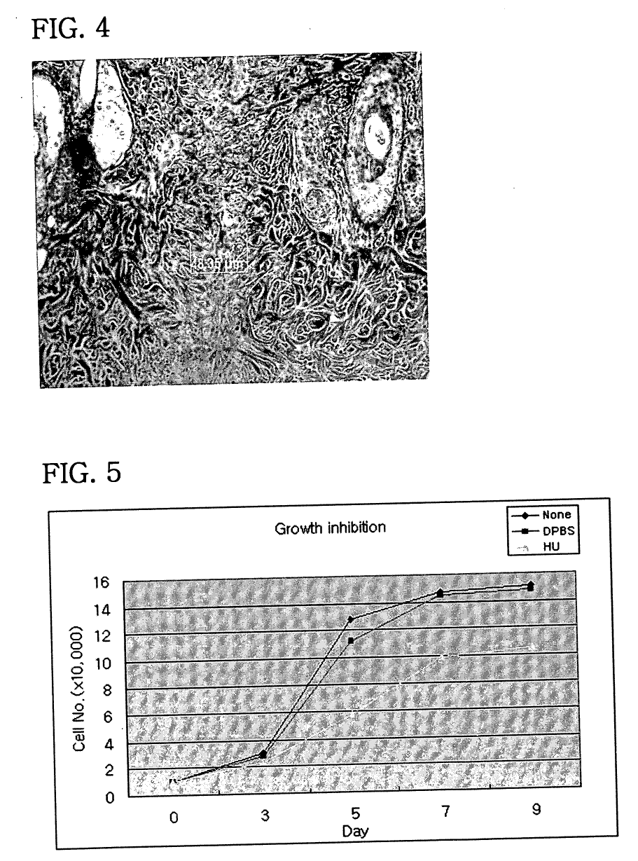

Nine female Sprague-Dawley rats weighing 280-300 g were shaved on their dorsal regions and divided into 3 groups (3 rats each group). As shown in FIG. 2, six dorsal sites were wounded (length 1 cm) with surgical scissors and sutured. Three wounds at the left side were treated with 0.5 cc/wound of the solution A and those at the right side were treated with 0.5 cc/wound of the solution B. Tissues were separated from the wound sites 2, 4, and 6 weeks after the injection, embedded into paraffin block, and MT stained. FIGS. 3 and 4 show the results obtained 2 weeks after treatment with solutions A and B, respectively.

It can be seen from the results that the wound sites treated with the solution B have substantially less scar, whereas the wound sites treated with the solution A have visible scars.

### Comparative Example 2

### Hyaluronic Acid Solution

Preparation

Solution A was DPBS (Dulbecco's Phosphate Buffer Saline, Invitrogen Corp.). Solution B was prepared by dissolving hyaluronic acid in the solution A.

Experimentation

In Vitro

WS1 cells that showed more than 95% viability in trypan blue test were selected, thawed from liquid nitrogen, and then grown in 75 cc flask (T75) to form 80% monolayer (about 6×10⁶/T75) on MEM medium (10% FBS). The detection was performed on 24-well plate at the concentration of 10,000 and was repeated three times on each sample. Each well (10⁴ cells/well) in a 24-well plate containing 3.6 ml of cell suspension was inoculated with DPBS solution A or hyaluronic acid solution B and cultured at 37° C. for 24 hours. The number of cells was counted using hemocytometer 3, 5, 7, and 9 days after the inoculation. The results are shown in FIG. 5.

It can be seen from FIG. 5 that the effect of the solution B on inhibiting cell growth is greater than that of the solution A.

In Vivo

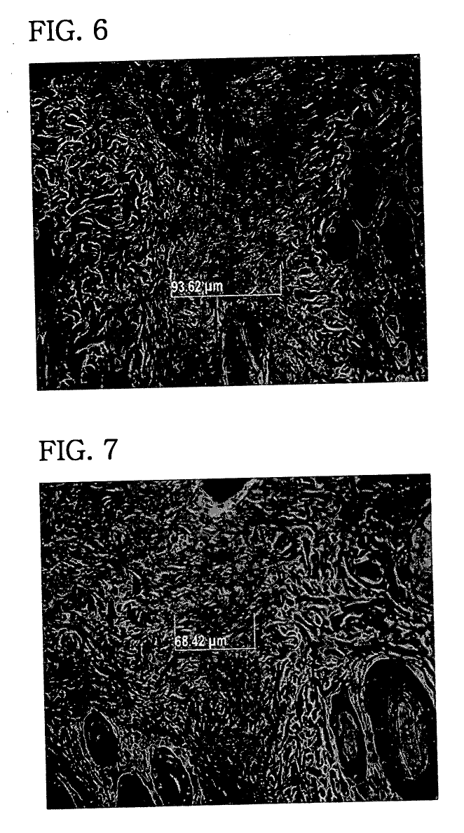

Nine female Sprague-Dawley rats weighing 280-300 g were shaved on their dorsal regions and divided into 3 groups (3 rats each group). As shown in FIG. 2, six dorsal sites were wounded (length 1 cm) with surgical scissors and sutured. Three wounds at the left side were treated with 0.5 cc/wound of the solution. A and those at the right side were treated with 0.5 cc/wound of the solution B. Tissues were separated from the wound sites 2, 4, and 6 weeks after the injection, embedded into paraffin block, and MT stained. FIGS. 6 and 7 show the results obtained 2 weeks after treatment with solutions A and B, respectively.

It can be seen from the results that the wound sites treated with the solution B have less scars, whereas the wound sites treated with the solution A have visibly much scars.

### Example 1

### Alkaline Hyaluronic Acid Solution (pH 8.5)

Preparation

Solution A was prepared by dissolving hyaluronic acid (Sigma Aldrich) in DPBS (Invitrogen Corp.) at a concentration of 25 mg/ml and adjusting the pH as 7.0 with the addition of 1N NaOH and 1N HCl. Solution B was prepared by adjusting the pH of the solution A as 8.5 with the addition of 1N NaOH and 1N HCl.

Experimentation

In Vitro

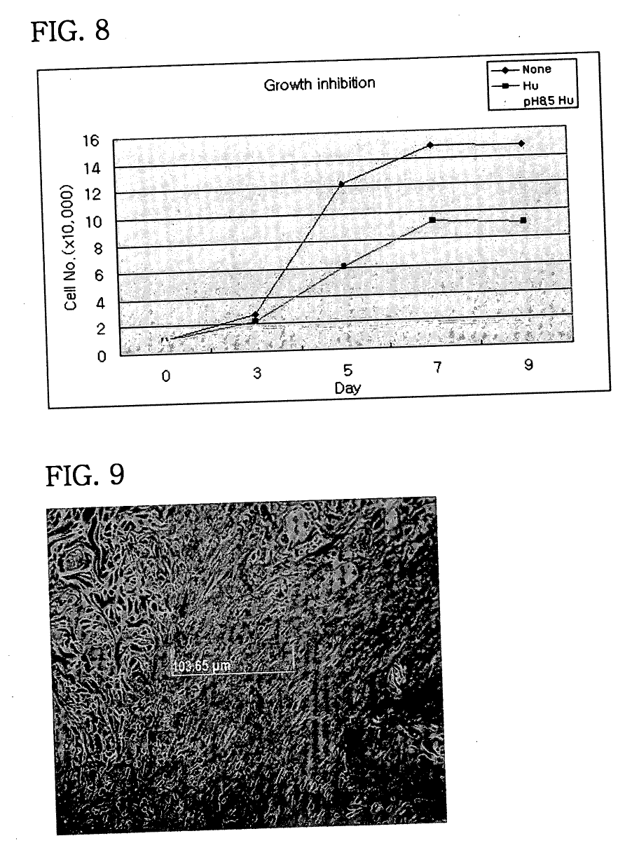

WS1 cells that showed more than 95% viability in trypan blue test were selected, thawed from liquid nitrogen, and then grown in 75 cc flask (T75) to form 80% monolayer (about 6×10⁶/T75) on MEM medium (10% FBS). The detection was performed on 24-we1l plate at the concentration of 10,000 and was repeated three times on each sample. Each well (10⁴ cells/well) in a 24-well plate containing 3.6 ml of cell suspension was inoculated with pH 7.0 hyaluronic acid solution A or pH 8.5 hyaluronic acid solution B and cultured at 37° C. for 24 hours. The number of cells was counted using hemocytometer 3, 5, 7, and 9 days after the inoculation. The results are shown in FIG. 8.

It can be seen from FIG. 8 that the effect of the solution B on inhibiting cell growth is highly greater than that of the solution A.

In Vivo

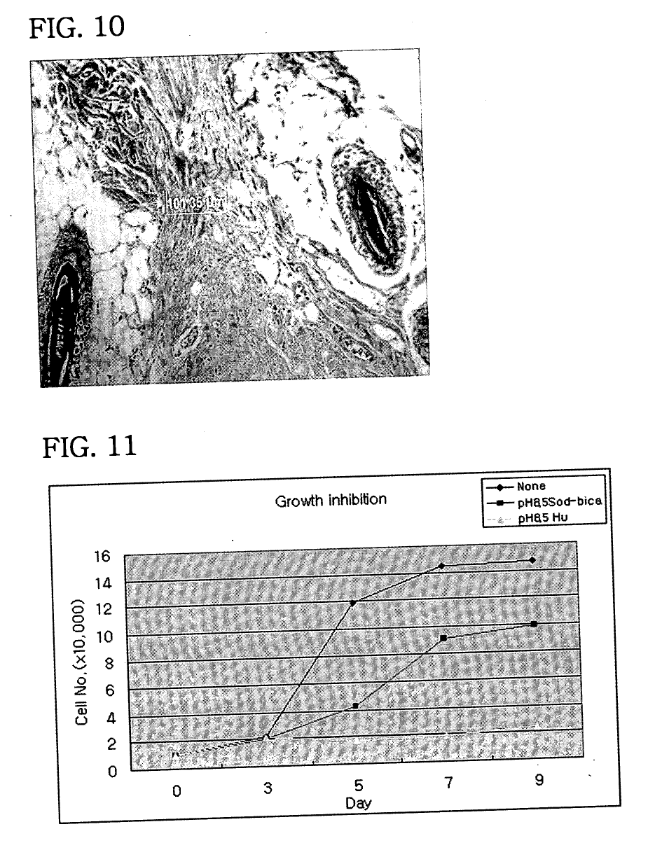

Nine female Sprague-Dawley rats weighing 280-300 g were shaved on their dorsal regions and divided into 3 groups (3 rats each group). As shown in FIG. 2, six dorsal sites were wounded (length 1 cm) with surgical scissors and sutured, Three wounds at the left side were treated with 0.5 cc/wound of the solution A and those at the right side were treated with 0.5 cc/wound of the solution B. Tissues were separated from the wound sites 2, 4, and 6 weeks after the injection, embedded into paraffin block, and MT stained. FIGS. 9 and 10 show the results obtained 2 weeks after treatment with solutions A and B, respectively.

It can be seen from the results that the wound sites treated with the solution B have substantially no scar, whereas the wound sites treated with the solution A have noticeable scars.

### Example 2

### Alkaline Hyaluronic Acid Solution (pH 8.5)

Preparation

Solution A was prepared by adjusting pH of 8.4% sodium bicarbonate injection solution (Jeil Pharma. Co., Ltd., Seoul, Korea) as 8.5 with the addition of 1N NaOH solution and 1N HCl solution. Solution B was prepared by dissolving hyaluronic acid (Sigma Aldrich) in DPBS (Invitrogen Corp.) at a concentration of 25 mg/ml and adjusting the pH as 8.5 with the addition of 1N NaOH and 1N HCl solution.

Experimentation

In Vitro

WS1 cells that showed more Man 95% viability in trypan blue test were selected, thawed from liquid nitrogen, and then grown in 75 cc flask (T75) to form 80% monolayer (about 6×10⁶/T75) on MEM medium (10% FBS). The detection was performed on 24-well plate at the concentration of 10,000 and was repeated three times on each sample. Each well (10⁴ cells/well) in a 24-well plate containing 3.6 ml of cell suspension was inoculated with pH 8.5 sodium bicarbonate solution A or pH 8.5 hyaluronic acid solution B and cultured at 37° C. for 24 hours. The number of cells was counted using hemocytometer 3, 5, 7, and 9 days after the inoculation. The results are shown in FIG. 11.

It can be seen from FIG. 11 that the effect of the solution B on inhibiting cell growth is highly greater than that of the solution A.

In Vivo

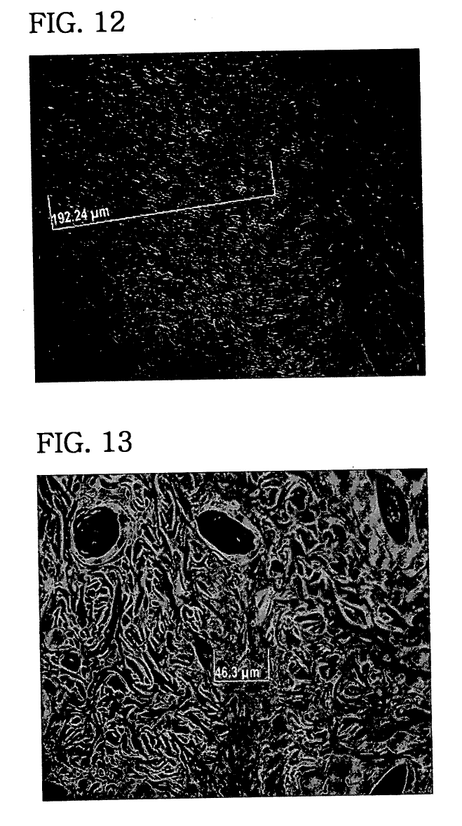

Nine female Sprague-Dawley rats weighing 280-300 g were shaved on their dorsal regions and divided into 3 groups (3 rats each group). As shown in FIG. 2, six dorsal sites were wounded (length 1 cm) with surgical scissors and sutured. Three wounds at the left side were treated with 0.5 cc/wound of the solution A and those at the right side were treated with 0.5 cc/wound of the solution B. Tissues were separated from the wound sites 2, 4, and 6 weeks after the injection, embedded into paraffin block, and MT stained. FIGS. 12 and 13 show the results obtained 2 weeks after treatment with solutions A and B, respectively.

It can be seen from the results that the wound sites treated with the solution B have substantially no scar, whereas the wound sites treated with the solution A have noticeable scars.

Consequently, it is evident that the composition consisting of alkalinizing agent and hyaluronic acid and having alkaline pH shows the synergism in inhibiting scar formation in wound region over each of hyaluronic acid composition having pHs less than 7 or alkalinizing agent individually. Therefore, the pharmaceutical composition of the present invention is ideal at the filed of the plastic surgery.

What is claimed is:

1. A pharmaceutical composition for inhibiting scar formation on wound region which consists of alkalinizing agent and hyaluronic acid and has alkaline pH.

2. The pharmaceutical composition of claim 1, wherein the pH of said composition is 8.5.

3. The pharmaceutical composition of claim. 1, wherein said alkalinizing agent is selected from the group consisting of sodium bicarbonate, NaOH and KOH.

4. The pharmaceutical composition of claim 2, wherein said alkalinizing agent is selected from the group consisting of sodium bicarbonate, NaOH and KOH.
